(19) United States
(12) Patent Application Publication
(10) Pub. No.: US 2017/0157083 A1
(43) Pub. Date: Jun. 8, 2017

(54) CONCENTRATED FELBAMATE FORMULATIONS FOR PARENTERAL ADMINISTRATION

(71) Applicant: Perosphere Inc., Danbury, CT (US)

(72) Inventors: Sasha H. Bakhru, Providence, RI (US); Bryan E. Laulicht, Danbury, CT (US); Edith Mathiowitz, Brookline, MA (US); Solomon S. Steiner, Mount Kisco, NY (US)

(21) Appl. No.: 15/438,530

(22) Filed: Feb. 21, 2017

Related U.S. Application Data
(63) Continuation of application No. 14/238,138, filed on Feb. 10, 2014.
(60) Provisional application No. 61/522,811, filed on Aug. 12, 2011.

(51) Int. Cl. A61K 31/27; A61K 9/14; A61K 9/00; A61K 47/20; A61K 9/08; A61K 47/10

(52) U.S. Cl. CPC A61K 31/27; A61K 9/08; A61K 47/10; A61K 9/0019; A61K 47/20; A61K 9/145

(57) ABSTRACT

Formulations of a neuroprotective agent for parenteral administration are described herein. The formulation is in the form of a concentrated (supersaturated) solution or a concentrated suspension of microparticles. The suspension medium or the solution solvent carrier may also contain dissolved neuroprotective agent. For the supersaturated solutions, the agent is dissolved a high concentrations of at least about 1% by weight, 5% by weight, 10% by weight, 15% by weight, or 20% by weight in a solvent suitable for parenteral administration. For the concentrated suspension, the microparticles have an effective particle size form about 100 nm to about 5 microns, preferably form about 50 nm to about 3 microns, more preferably from about 10 nm to about 2 microns. The formulations described herein can be used to treat a variety of neurological disease/disorders and/or neurological injury or trauma.

### CROSS-REFERENCE TO RELATED APPLICATION

This application claims priority to U.S. Provisional application No. 61/522,811, filed Aug. 12, 2011. The disclosure of which is incorporated herein by reference.

### FIELD OF THE INVENTION

This invention is in the field of formulations of neuroprotective agents, such as anti-convulsant and/or anti-epileptic agents, for the treatment of neurological disorders, damage, and/or injury, particularly concentrated solutions and suspensions of carbamazepine, felbamate, and fluorofelbamate.

### BACKGROUND OF THE INVENTION

Neurological damage following acute cell death in the brain is a tremendous health burden without any approved pharmacological intervention. Stroke is the leading cause of serous, long-term disability in the United States. Additionally, according to the Centers for Disease Control and Prevention nearly 1.4 million people in the United States sustain a traumatic brain injury. Also, according to the National Cancer Institute there are approximately 20 thousand new cases of brain cancer each year in the United States alone. Stroke and traumatic brain injury directly cause neuronal cell death. Acute treatments for brain cancer including but not limited to surgical resection and radiation therapy, also yield neuronal cell death.

When neurons are destroyed they release their contents, including large amounts of the excitatory neurotransmitter glycine, into the extracellular fluid in high concentrations. Elevated extracellular glycine levels interact with the strychnine insensitive glycine receptor to open their attached calcium ion channels. In keeping with the concentration gradient, calcium ions traverse the open ion channels letting large quantities of calcium into the neuronal cells. Once inside, in high intracellular concentrations calcium triggers the proteolytic activity of calpain to break down to an untoward degree, which can lead to cellular injury or cell death.

Seizures occur when neurons exhibit aberrant action potentials. Felbamate is used to treat epileptic seizures by reducing calcium influx, which would otherwise tend to depolarize neurons and increase their propensity to trigger an action potential. Felbamate can stop or reduce the severity of seizures.

However, repeated administration of felbamate, particularly over extended periods of time, can result in sever side effects, such as aplastic anemic and/or liver damage, which can be fatal. Reports have estimated the risk of developing aplastic anemia due to oral administration of felbamate once daily for at least 25 days is 1:3,600 and 1:5,000, of which 30% of the cases are fatal. In the liver, felbamate can be converted to a short-lived toxic metabolite that is believed to be responsible for the observed adverse effects. Fluorofelbamate was developed to avoid the untoward hepatic metabolic pathway of felbamate. For many of the above described neuroprotective indications, patients are unconscious, which makes oral administration challenging if not impossible. Therefore, parenteral formulations are preferred for these therapeutic applications. As well, parenteral formulations avoid first pass metabolism, which lessens the likelihood of reaching toxic concentrations of the undesirable hepatic metabolite of felbamate. However, parenteral formulations administered outside of the vasculature exhibit a lag in absorption time before the blood levels rise to a therapeutic concentration, which can result in further neuronal damage.

There exists a need for formulations of neuroprotective agents, such as the anti-convulsant agents felbamate and fluorofelbamate, which can be administered intravenously in a single administration or over a short period of time, thereby minimizing the potential for adverse side effects associated with felbamate, and which deliver the agent rapidly into circulation in order to reduce, minimize or prevent secondary neuronal damage.

Therefore, it is an object of the invention to provide formulations of neuroprotective agents, which can be administered parenterally in a single or few repeat administrations or over a short period of time, thereby minimizing the adverse side effects associated with these compounds, and minimizing lag time between neuronal damage and achieving therapeutic concentrations to prevent further neuronal damage.

It is a further object of the invention to provide methods and formulations for treating or preventing epileptic seizures.

It is a further object of the invention to provide improved methods and formulations for the treatment of neurological disorders.

### SUMMARY OF THE INVENTION

Formulations of a neuroprotective agent (e.g., anti-convulsant and/or anti-epileptic agent, such as carbamazepine, felbamate, and fluorofelbamate) for parenteral administrations are described herein. In one embodiment, the formulation is in the form of microparticles of the agent(s) suspended in a pharmaceutically acceptable carrier suitable for parenteral administration. The microparticles can be prepared by dissolving the agent(s) in a solvent, with or without heating, and then adding the solution of the agent to a non-solvent, with or without cooling. In some embodiments, the solvent is an organic solvent. In other embodiments, the solvent is water or an aqueous solvent, particularly heated water or a heated aqueous solvent. In one embodiment, the solvent is an organic solvent and the non-solvent is water or an aqueous solvent. In another embodiment, the solvent is heated water of an aqueous solvent and the non-solvent is water or an aqueous solvent.

The non-solvent generally contains a surface modifying agent. In particular embodiments, the surface modifying agent is a surfactant. In preferred embodiments, the surfactant has a hydrophilic-lypophilic balance (HLB) of at least about 15, preferably greater than 15. In some embodiments, the surfactant has an HLB of at least about 15, preferably greater than 15 and is a non-ionic surfactant.

After formation of the microparticles, the microparticles can be isolate, dried, and stored until use. In these embodiments, if the non-solvent contains a surface modifying agent, such as a surfactant, the surface modifying agent is incorporated into, onto, and/or dispersed throughout the microparticles. If the surface modifying agent is a solid at ambient conditions, or the surface modifying agent is removed prior to final formulation, the microparticles are typically in the form of a dry powder. If the surface modifying agent is a liquid at ambient conditions, the microparticles are typically in the form of a slurry. The microparticles can be reconstituted in an appropriate carrier prior to administration. The carrier way contain one or more pharmaceutically acceptable excipients including the surfactant(s). The carrier may also contain dissolved neuroprotective agent (e.g., carbamazepine, felbamate, and fluorofelbamate).

In other embodiments, the microparticles can be administered immediately upon or after formation. For example, if the solvent is sterilized, heated water or aqueous solvent and the non-solvent is (sterilized) water or an aqueous solution, particularly cooled water or aqueous solution, upon mixing, particles form due to the differences in temperature of the solvent and non-solvent. The particles are suspended in the water or aqueous solution, which is suitable for parenteral administration. The suspending medium can optionally contain dissolved neuroprotective agent.

The microparticles have an effective panicle size from about 100 nm to about 5 microns, preferably from about 50 nm to about 3 microns, more preferably from about 10 nm to about 2 microns. In particular embodiments, the particle size distribution is at least 80% of the particles by volume have the particle size ranges above.

In still other embodiments, the formulation is in the form of a supersaturated solution of the anti-convulsant and/or anti-epileptic agent. In some embodiments, the drug is dissolved at high concentrations of at least about 1% by weight, 5% by weight, 10% by weight, 15% by weight, or 20% by weight in a solvent suitable for parenteral administration. In particular embodiments, the agent is dissolved in a polyethylene glycol, such as PEG 300, PEG 400, PEG 600, glycerin, propylene glycol, sorbitol, ethylene glycol, or a surfactant, such as polysorbate 20. The resulting supersaturated solution is stable (e.g., no precipitation) for at least one hour, two hours, three hours, four hours, six hours, eight hours, 12 hours, 24 hours, 30 hours, 36 hours, or 48 hours. In preferred embodiments, the resulting supersaturated solution is stable for at least one week, one month, or one year. Prior to administration, the concentrated solution cats be diluted in one or more solvent suitable for parenteral administration, such as water, antimicrobial agents, ethanol, propylene glycol, and combinations thereof.

The formulations described herein can be used to treat a variety of neurological diseases/disorders and/or neurological injury or trauma. Exemplary diseases or disorders include, but are not limited to, preventing/reducing seizures, stroke, traumatic brain injury, brain tumor resection, brain tumor irradiation, bipolar disorder, trigeminal neuralgia, attention-deficit hyperactivity disorder (ADHD), schizophrenia, phantom limb syndrome, complex regional pain syndrome, paroxysmal extreme pain disorder, neuromyotonia, intermittent explosive disorder, and post-traumatic stress disorder.

In certain embodiments, the formulations described herein are used to treat/prevent seizures and/or other neurological damage, such as stroke, traumatic brain injury, and/or brain tumor resection/irradiation, where rapid delivery of the active agent is required to prevent further damage arising from neuronal injury. In certain embodiments, the formulations described herein are used to prevent secondary neuronal damage accompanying local or global neuronal cell injury or death. For example, the formulations described herein can be used to prevent seizures and/or reduce the length and/or severity of seizures.

The formulations are administered to provide an effective amount of the active agent. For example, suitable amount of the suspensions and/or solutions are administered to provide a dose of the active agent ranging from 100-2000 mg, preferably 200-1000 mg, more preferably 400-600 mg. However, the appropriate dosage can be determined by the attending physician based on a variety of factors including age and weight of the patient and diseases or disorder to be treated.

### DETAILED DESCRIPTION OF THE INVENTION

### I. Definitions

“Microparticle,”, as used herein, refers to any shaped panicle with at least one dimension in the range of 10 nanometers to 1,000 microns.

“Neuroprotective”, as used herein refers to any agent that reduces brain cell damage subsequent to primary neuronal cell death.

“Anticonvulsant”, as used herein, refers to any agent that reduces the severity of a seizure.

“Intravenously injectable”, as used herein, refers to any formulation that is capable of being injected into the circulatory system of a mammal.

“Diluent”, as used herein refers to an agent that when introduced reduces the concentration of another agent.

“Slurry”, as used herein, refers to any viscous suspension.

“Neuronal”, as used herein, refers to pertaining to the brain.

“Primary neuronal injury” refers to cell injury or death directly resulting from a pathophysiology.

“Secondary neuronal cell death” refers to cells that die subsequent to a primary neuronal injury.

“Non-solvent” refers to any poor solvent for an agent which is incapable of dissolving more than 1 milligram of the agent in 1 milliliter of the non-solvent.

“Effective particle size” refers to the diameter of a circle with equivalent area to that of the particulate shape.

“Supersaturated” refers to solutions that contain a greater quantity of a solute at a given temperature than they would without an additional processing step, such as heating.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

### II. Formulations

A. Neuroprotective Agents

The compositions described herein contain one or more neuroprotective agents, such as anticonvulsant agents and/or anti-epileptic agents. Suitable agents include, but are not limited to, such as carbamazepine, felbamate, fluorofelbamate.

Carbamazepine (CBZ) is an anticonvulsant and mood-stabilizing drug used primarily is the treatment of epilepsy and bipolar disorder, as well as trigeminal neuralgia. It is also used off-label for a variety of indications, including attention-deficit hyperactivity disorder (ADHD), schizophrenia, phantom limb syndrome, complex regional pain syndrome, paroxysmal extreme pain disorder, neuromyotonia, intermittent explosive disorder, and post-traumatic stress disorder.

Felbamate (marketed under the brand name Felbatol by Meda Pharmaceuticals Inc.) is an anticonvulsant drug used in the treatment of epilepsy. It is used to treat partial seizures (with and without generalization) in adults and partial and generalized seizures associated with Lennox-Gastaut syndrome in children. However, an increased risk of potentially fetal aplastic anemia and/or liver failure, due to repealed administration over an extended period of time, has limited its usage to severe refractory epilepsy. Felbamate is an inhibitor of CYP2C19, an isoenzyme of the cytochrome P450 system involved in the metabolism of several commonly used medications. Felbamate interacts with several other anti-epileptic drugs (AEDs), including phenytoin, valproate, and carbamazepine; dosage adjustments may be necessary to avoid adverse effects. Concomitant administration of felbamate and carbamazepine decreases blood levels of both drugs, while increasing the level of carbamazepine-10, 11 epoxide, the active metabolite of carbamazepine.

Fluorofelbamate is a derivative of felbamate that was developed to overcome the life-threatening toxicity of felbamate. Fluorofelbamate lacks the reactive intermediate (glutathionine-aldehyde adduct) characterized in felbamate toxicity. In specific embodiments, fluorofelbamate is provided in a parenteral formulation.

The agent can be used as the free acid or free base or as a pharmaceutically acceptable salt. “Pharmaceutically acceptable salt”, as used herein, refer to derivatives of the compounds defined by Formula I, II, and III wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, form non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use, ” P. Heinrich Stahl and Camille G. Wermuth, Eds., Wiley-VCH, Weinheim, 2002.

The compounds described herein may have one or more chiral centers and thus exist as one or more stereoisomers. Such stereoisomers can exist as a single enantiomer, a mixture of diastereomers or a racemic mixture.

As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g., Jacques, J. et al., “Enantiomers, Racemates, and Resolutions”, John Wiley and Sons, Inc. 1981).

Suitable dosages of the active agent are 100-2000 mg, preferably 200-1000 mg, more preferably 400-600 mg. However, the appropriate dosage can be determined by the attending physician based on a variety of factors including age and weight of the patient and diseases or disorder to be treated.

The formulations can contain one or more additional active agents that are appropriate to be administered with neuroprotective agents.

B. Concentrated Suspensions

In one embodiment, the formulation is the form of a concentrated suspension or slurry. The suspension can be prepared immediately prior to use. For example, as discussed below, microparticles can be prepared by adding a heated aqueous solution of the neuroprotective agent (e.g., anticonvulsants, such as carbamazepine, felbamate, and fluorofelbamate) to an excess of lower temperature sterile water or aqueous solution, such as an aqueous surfactant solution. The resulting microparticles are suspended in an aqueous medium, which can be administered immediately to the patient.

In other embodiments, the microparticles are prepared, isolated, and dried and stored under appropriate conditions. The microparticles can be reconstituted in an appropriate pharmaceutically acceptable carrier prior to administration.

The suspending medium optionally contains dissolved neuroprotective agent (e.g., anticonvulsants, such as carbamazepine, felbamate, and fluorofelbamate).

a. Microparticles

The microparticles formed by the methods described herein have reduced crystallinity compared to stock, non-micronized anti-convulsant and/or anti-epileptic agent. The microparticles have an effective particle size of less then about 100 microns. The microparticles preferable have an effective particle size from about 100 nm to about 5 microns, preferably from about 50 nm to about 3 microns, more preferably from about 10 nm to about 2 microns. In particular embodiments, the particle size distribution is at least 80% of the particles by volume have the preferred particle size ranges listed above. The microparticles are rounded, ellipsoidal, and/or spherical.

In some embodiments, the isolated microparticles contain one or more surface modifying agents, preferably surfactants, incorporated into, onto, and/or dispersed throughout the drug particles. Preferably the microparticles contain one or more surface modifying agents adsorbed onto their surface. The surface modifying agent may be present is as amount ranging from 0.0001 to 90% by weight of the total weight of the surface modifying agent and the neuroprotective agent. In some embodiments, the surface modifying agent, preferably surfactant, is a solid at ambient temperature so that the microparticles are in the form of a powder. In other embodiments, the surface modifying agent, preferably surfactant, is a liquid at ambient temperature so that the microparticles form a slurry after isolation from the solvent.

i. Surfactants

A variety of surfactants can be used to prepare the microparticles and/or suspensions thereof. Surfactants can be classified as anionic, cationic, amphoteric, and nonionic surfactants and include phospholipids.

Examples of suitable anionic surfactants include, but are not limited to, sodium, potassium, and ammonium salts of long chain alkyl sulfontes and alkly aryl sulfonates suck as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate, and sodium deoxycholate.

Examples of suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine.

Examples of suitable nonionic surfactants include, but are not limited to, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates (TWEENS®), polyoxyethylene octylphenylether, PEG-1000 cetyl ether, polyoxyethylene tridecyl ether, polypropylene glycol butyl ether, POLOXAMER®401, stearoyl monoisopropanolamide, and polyoxyethylene hydrogenated tallow amide.

Examples of amphoteric surfactants include, but are not limited to, sodium N-dodecyl-β-alanine, sodium N-lauryl-β-iminodipropionate, myristoamphoacetate, lauryl betaine and lauryl sulfobetaine.

Suitable phospholipids include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phosphatidylcholines include such as dioleoylphosphatidylcholine, dimyristoylphosphatidylcholine (DMPC), dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoyl-phosphatidylcholine (DTPC), dilignoceroylphatidylcholine (DLPC); and phosphatidylethanolamines such as dioleoylphosphatidylethanolamine or 1-hexadecyl-2-palmitoylglycerophospho- ethanolamine. Synthetic phospholipids with asymmetric acyl chains (e.g., with one acyl chain of 6 carbons and another acyl chain of 12 carbons) may also be used.

Examples of phosphatidylethanol-amines include, but are not limited to, dicaprylphosphatidylethanolamine, dioctanoylphosphatidyl-ethanolamine, dilauroylphosphatidylethanolamine, dimyristoylphosphatidyl-ethanolamine (DMPE), dipalmitoylphosphatidylethanolamine (DPPE), dipalmitoleoylphosphatidylethanolamine, distearoylphosphatidylethanolamine (DSPE), dioleoylphosphatidylethanolamine, and dilineoylphosphatidylethanol-amine.

Examples of phosphatidylglycerols include, but are not limited to, dicaprylphosphatidylglycarol, dioctanoylphosphatidylglycerol, dilauroylphosphatidylglycerol, dimyristoylphosphatidylglycerol (DMPG), dipalmitoylphosphatidylglycerol (DPPG), dipalmitoleoylphosphatidylglycerol, distearoylphosphatidylglycerol (DSPG), dioleoylphosphatidylglycerol, and dilineoylphosphatidylglycerol.

In a preferred embodiment, the surfactant is a polysorbate. In one embodiment, the surfactant has HLB of at least 15, preferably greater than 15. In other embodiments, the surfactant has an HLB of at least 15, preferably greater than 15 and is a non-ionic surfactant. In one embodiment, the surfactant is a polysorbate. In a preferred embodiment, the surfactant is polysorbate 20.

The suspension can contain one or more pharmaceutically acceptable excipients including, but not limited to, pH modifying agents, dispersing agents, tonicity modifying agents, plasticizers, crystallization inhibitors, wetting agents, bulk filling agents, bioavailability enhancers, and combinations thereof.

C. Concentrated Supersaturated Solutions

In other embodiment, the formulation is in the form of a concentrated solution. In some embodiments, the drug is dissolved at high concentrations of at least about 1% by weight, 5% by weight, 10% by weight, 15% by weight, or 20% by weight in a solvent suitable for parenteral administration. In particular embodiments, the neuroprotective agent (e.g., anticonvulsants, such as carbamazepine, felbamate, and fluorofelbamate) is dissolved in one or a combination of a polyethylene glycol, such as PEG 300, PEG 400, PEG 600, glycerin, propylene glycol, sorbitol, ethylene glycol, or a surfactant, such as polysorbate 20. The resulting supersaturated solution is stable (e.g., no precipitation) for at least one hour, two hours, three hours, four hours, six hours, eight hours, 12 hours, 24 hours, 30 hours, 36 hours, or 48 hours. Prior to administration, the concentrated solution can be diluted in one or more solvents suitable for parenteral administration, such as water, antimicrobial agents, ethanol, propylene glycol, and combinations thereof.

The solution can contain one or more pharmaceutically acceptable excipients including, but not limited to, pH modifying agents, tonicity modifying agents, plasticizers, crystallization inhibitors, wetting agents, bulk filling agents, bioavailability enhancers, and combinations thereof. The diluting solvent may contain one or more surfactants, such as those described above.

### III. Methods of Making

A. Concentrated Microparticle Suspensions

In some embodiments, a neuroprotective agent (e.g., anticonvulsants, such as carbamazepine, felbamate, and fluorofelbamate) is dissolved in a suitable solvent or solvent mixture. In some embodiments, the solvent or solvent mixture is water or an aqueous solvent. In other embodiments, the solvent or solvent mixture is an organic solvent. Suitable organic and aqueous solvent include, but are not limited to, dimethyl sulfoxide, heated water, glycerin and mixture thereof.

The neuroprotective agent (e.g., anticonvulsants, such as carbamazepine, felbamate, and fluorofelbamate) solution is then introduced into an excess of a non-solvent for the neuroprotective agent, which is miscible with the solvent. Suitable non-solvents include, but not limited to water, an aqueous solution of a surfactant (see the surfactants described above), and an aqueous surfactant (see the surfactants described above) solution containing dissolved neuroprotective agent. In some embodiments, the aqueous receiving solution is stirred. When the solvent mixes with the non-solvent, the mixture presents unfavorable solubility conditions for the neuroprotective agents (e.g., anticonvulsants, such as carbamazepine, felbamate, and fluorofelbamate) causing it to leave solution creating a particulate suspension.

In particular embodiments, the resultant particle size distribution is at least eighty volume percent between 100 nanometers and five microns in effective particle size, more preferably between fifty nanometers and three microns in effective particle size, and most preferably between 10 nanometers and two microns in effective particle size.

In embodiments employing an organic solvent, the particle suspension can be stirred, in the presence of absence of heating and/or vacuum, until a sufficient quantity of the organic solvent has evaporated to effect particle formation.

In particular embodiments, the non-solvent contains a surface modifying agent, such as a surfactant. In some embodiments, the surfactant has a hydrophilic lipophilic balance (HLB) at least about fifteen. In more particular embodiments, the surfactant has an HLB of greater than 15. Suitable surfactants include, but are not limited to, polysorbate 20. The concentration of surfactant during particle formation is generally greater than 0.05 weight per volume percent, more preferably greater than 0.1 weight per volume percent, and most preferably greater than 0.4 weight per volume percent. However, the concentration can be lower or greater than these values dependent on the solvent, non-solvent, and surfactant that are used.

In some embodiments, stock felbamate, fluorofelbamate, or other carbamazepine powder is suspended in an aqueous surfactant solution. The aqueous felbamate suspension is then heated to at least approximately 50°, preferably to at least approximately 60°, and more preferably to at least approximately 70° Celsius until the felbamate dissolves. The heated felbamate solution is then allowed to cool in the presence or absence of an external cooling element and in the presence or absence of stirring. As the temperature decreases, the felbamate precipitates from solution to form microparticles. In preferred embodiments, the resultant felbamate particle size distribution is at least eighty volume percent between 100 nanometers and five microns in effective particle size, more preferably between 50 nanometers and three microns in effective particle size, and most preferably between 10 nanometers and two microns in effective particle size. In particle embodiments, the particle size distribution is at least 80% of the particles by volume have the particle size ranges above.

In embodiments in which the particle size is unstable, the resultant felbamate suspension can by rapidly frozen by any one or a combination of the following including, but not limited to, electronic refrigeration, introduction onto dry ice, and introduction into liquid nitrogen. The frozen suspension can by lyophilized to produce felbamate microparticle slurry in the remaining surfactant, provided the surfactant is liquid in ambient condition. In some embodiments, the surfactant is a solid in ambient conditions thereby creating a dry powder after lyophilization.

In preferred embodiments, the concentration of surfactant in solution prior to drying is reduced such that when the resultant suspension is lyophilized, it produces a dry powder. The resultant slurry or dry powder can be resuspended to create a concentrated felbamate microparticle suspension for parenteral administration or stored as a two part suspension for parenteral administration after resuspension.

1. One Part Suspensions

In embodiments that produce dilute suspensions of the neuroprotective agent (e.g., anticonvulsants, such as carbamazepine, felbamate, and fluorofelbamate), concentration of the suspension can be achieved by any or a combination of the following including, but not limited to, centrifugation, decanting, and resuspension in a lesser volume; drying by means of lyophilization, spray drying, air drying, or other means, followed by resuspension in a lesser volume; and reduction in the volume of the suspension media using a spin column. In preferred embodiments, the resultant suspension concentration is approximately or greater than five weight percent, more preferably approximately or greater than ten weight per volume percent, and most preferably approximately or greater than twenty weight per volume percent. In some preferred embodiments, the suspending media is one or a combination of the following including, but not limited to, water for injection, sterile phosphate buffered saline, a sterile aqueous surfactant solution, and a sterile aqueous antimicrobial solution.

2. Two Part Suspensions

In embodiments producing a microparticle slurry or dry powder, the resultant slurry or dry powder formulation can be stored separately from its suspending media until administration. In some embodiments the slurry or dry powder can be stored separately from the resuspension media in separate containers. In preferred embodiments the slurry or dry powder is stored dry within one compartment of a two-compartment syringe. The resuspension media is stored in a separate compartment within the syringe. Prior to administration, the slurry or powder is resuspended in the resuspension media for administration as a single suspension.

B. Concentrated Solutions

In some embodiments, the neuroprotective agent (e.g., anticonvulsants, such as carbamazepine, felbamate, and fluorofelbamate) is dissolved at high concentrations, e.g. greater than about one weight percent, preferably greater than about five weight percent, and more preferably greater than about ten weight percent, in a solvent suitable for parenteral administration (e.g., injection), more preferably suitable for intravenous injection.

In a specific embodiment, the agent is added above its solubility limit in one or more solvents. The gent is typically added to the one or more solvents. Suitable solvents include, but are not limited to, polyethylene glycol 300, polyethylene glycol 400, and polyethylene glycol 600. The solution of the agent is heated, for example to a temperature of at least about 50° C., preferably at least 60° C., and more preferably at least about 70° C. until the felbamate, fluorofelbamate, or carbamazepine dissolves and is then cooled, for example to room temperature, while remaining in solution to form a stable, supersaturated solution. In this embodiment, the resultant supersaturated solution remains in solution at room temperature for at least one hour where the concentration of agent in the supersaturated solution is at least 5%, preferably at least 10%, more preferably at least 15%, most preferably 20% weight per volume. The upper limit for the concentration of the agent in the supersaturated solution preferably is less than 35% weight by volume, less than 20% weight by volume, or less than 15% weight by volume. In preferred embodiments the agent in the super saturated solution is felbamate or fluorofelbamate.

In certain embodiments, the supersaturated solution remains stable for at least one week, one month, or one year.

In another embodiment, the agent is dissolved in glycerin heated to above approximately 100° C. and then cooled to ambient storage temperatures to form a supersaturated solution. Since felbamate and fluorofelbamate have a solubility in water of less than one milligram per milliliter, the ability to create a stable supersaturated solution of felbamate in an intravenously acceptable solvent is unexpected.

In some embodiments, felbamate is dissolved in heated polyethylene glycol 300, polyethylene glycol 400, polyethylene glycol 600, propylene glycol, sorbitol, ethylene glycol, or polysorbate 20 in concentrations greater than would enter solution in water at 25° C.

In some embodiments, prior to injection, the solution of the agent can be diluted with another injectable solvent including, but not limited to, water, one or more antimicrobial agents, ethanol, and propylene glycol, and combinations thereof.

### IV. Methods of Use

The formulations described herein can be used to treat a variety of neurological diseases/disorders and/or to prevent secondary neuronal injury following neurological hypoxia, injury or trauma. Exemplary diseases or disorders include, but are not limited to, preventing/reducing seizures, stroke, traumatic brain injury, brain tumor resection, brain tumor irradiation, bipolar disorder, trigeminal neuralgia, attention-deficit hyperactivity disorder (ADHD), schizophrenia, phantom limb syndrome, complex regional pain syndrome, paroxysmal extreme pain disorder, neuromyotonia, intermittent explosive disorder, and post-traumatic stress disorder.

In certain embodiments, the formulations described herein are used to treat/prevent seizures, status epilepticus and/or other neurological damage, such as stroke, traumatic brain injury, and/or brain tumor resection/irradiation, where rapid delivery of the active agent is required to prevent further damage arising from neuronal injury. For example, the formulations described herein can be used to prevent seizures and/or reduce the length and/or severity of seizures.

Felbamate has been used to treat or prevent neurological diseases and/or injury. However, long-term felbamate administration can result in aplastic anemic, a sometime fatal side effect. The risk of aplastic anemia associated with cronic oral felbamate dosing has been reported as between 1:3,600 and 1:5,000, of which 30% of the cases are fatal.

In contrast, the suspensions and solutions described herein are administered parenterally as a single administration or a short course of treatment which is less than 48 hours in duration, preferably less than 8 hours, more preferably less than 6 hours. Aplastic anemia has not been shown to develop after a single administration of felbamate. The formulations described herein provide rapid delivery of the active agent to prevent further damage resulting from neurological injury or damage.

The formulations are administered to provide an effective amount of the active agent. For example, suitable amount of the suspensions and/or solutions are administered to provide a dose of the active agent ranging from 100-2000 mg, preferably 200-1000 mg, more preferably 400-600 mg. However, the appropriate dosage can be determined by the attending physician based on a variety of factors including age and weight of the patient and diseases or disorder to be treated.

### EXAMPLES

### Example 1

### Preparation of Microparticles Using Organic Solvent Method

Felbamate was dissolved in dimethyl sulfoxide to create a five weight per volume percent solution. The felbamate solution was introduced into one hundred times the volume of a one weight per volume percent polysorbate 20 aqueous solution stirred at 1,500 revolutions per minute using an overhead mixer equipped with an impeller blade. The resultant microparticles of felbamate were imaged using SEM. The particles appeared round in shape, suitable for injection. Under the same experimental conditions. Span 80 was substituted for polysorbate 20. The felbamate formed crystals, which are unsuitable for injection.

Felbamate was dissolved in glycerin heated above 120° C. to create a 5% weight per volume solution. The felbamate solution was introduced into one hundred times the volume of a one weight per volume percent polysorbate 20 aqueous solution stirred at 1,500 revolutions per minute using an overhead mixer equipped with an impeller blade. The resultant microparticles of felbamate were imaged using SEM. The particles appeared round in morphology and were significantly smaller, on the order of microns as compared to hundreds of microns for native felbamate powder.

### Example 2

### Washing and Resuspension of Microparticles

Felbamate microparticles were centrifuged in a refrigerated centrifuge spinning at 8,000 revolutions per minute for a period of twenty minutes. The supernatant was substantially removed enabling the felbamate microparticles to be resuspended in any desired media.

### Example 3

### Preparation of Felbamate Microparticles Using the Aqueous Solvent Method

Felbamate was dissolved in water heated to 90° C. to create a five weight per volume percent solution. The felbamate solution was introduced into one hundred times the volume of a one weight per volume percent polysorbate 20 aqueous solution stirred at 1,500 revolutions per minute using an overhead mixer equipped with an impeller blade.

### Example 4

### Preparation of Felbamate Microparticles in the Presence of a Surface Modifying Agent

Felbamate was dissolved in an aqueous solution of polysorbate 20 having a concentration of one weight percent and heated to 90° C. The solution was then cooled to allow the felbamate to precipitate, thereby forming microparticles.

Example 5

### Drying Felbamate Suspensions

Vessels containing aqueous or predominantly aqueous suspensions of felbamate were introduced into a dewar containing liquid nitrogen to rapidly freeze the suspension. The frozen suspension was then lyophilized to produce either a felbamate slurry or dry powder.

### Example 6

### Felbamate Supersaturated Solution

Felbamate was added at a concentration of up to 20 weight per volume percent to polyethylene glycol 300 and polyethylene glycol 400. Solutions were heated above 70° C. after which the felbamate dissolved. Upon cooling the samples in ambient conditions to room temperature, which was approximately 25° C., samples up to approximately 12.5 weight per volume percent remained in solution for at least eight months. The sample also remained to solution when heated or cooled to 40° C., 4° C., and −20° C. for 30 minutes and then allowed to return to 25° C.

### Example 7

### Felbamate Supersaturated Solution Mixtures

Felbamate was added at a concentration of up to 10 weight per volume percent in 65 volume per volume percent polyethylene glycol 300, 20 volume per volume percent polyethylene glycol 400, and 15 volume per volume percent propylene glycol. In a separate experiment, felbamate was added at a concentration of up to 10 weight per volume percent in 65 volume per volume percent polyethylene glycol 300, 20 volume per volume percent polyethylene glycol 400, 10 volume per volume percent propylene glycol, and 5 volume per volume percent polyethylene glycol 600.

Solutions were heated above 70° C. after which the felbamate dissolved. Upon cooling the samples in ambient conditions to room temperature, e.g., approximately 25° C., the sample remained in solution for at least 1 day.

### Example 8

### Felbamate Solution Injection

A supersaturated 10 weight per volume percent felbamate solution in PEG 300 was injected into 37° C. phosphate buffered saline. No solid particles were observed. Additional felbamate supersaturated solution at the same concentration was added 10 microliters at a time without forming solid particles up to at least 50 microliters. This indicates that felbamate will remain in suspension in blood plasma well in excess of the standard therapeutic doses of 400 to 600 mg per dose.

### Example 9

### Fluorofelbamate Supersaturated Solution

Fluorofelbamate was added at a concentration of 10 weight per volume percent to polyethylene glycol 300. Solutions were heated above approximately 80° C. after which the fluorofelbamate dissolved. Upon cooling the samples in ambient conditions to room temperature, which was approximately 25°, the sample remained in solution for at least 1 month.

1. A pharmaceutical composition comprising a 2 to 20 weight percent supersaturated solution of felbamate or fluorofelbamate in a non-aqueous pharmaceutically acceptable carrier suitable for parenteral administration.

2-6. (canceled)

7. The composition of claim 1, wherein the concentration of the felbamate or florofelbamate is between 5% weight by volume, and 12.5% by weight by volume.

8. The composition of claim 1, wherein the concentration of the felbamate or fluorofelbamate is between 1% by weight by volume and 35% weight by volume.

9. The composition of claim 1, wherein the room temperature stability of the supersaturated solution is greater than 1 month.

10. The composition of claim 1, wherein the solvent for the solution is a polyethylene glycol.

11. The composition of claim 10, wherein the polyethylene glycol is polyethylene glycol 300, polyethylene glycol 400, polyethylene glycol 600, or a mixture of any combination of at least two solvents selected from the group consisting of polyethylene glycol 300, polyethylene glycol 400, polyethylene glycol 600, and glycerin.

12-14. (canceled)

15. The composition of claim 1, wherein the felbamate or fluorofelbamate is a concentrated suspension of microparticles having a surface modifying agent adsorbed on the surface thereof, and an effective particle size of less than about 100 microns.

16. The composition of claim 15, wherein the concentration of the particles in the suspension is between 5% weight by volume and 20% weight by volume.

17-21. (canceled)

22. The composition of claim 1, wherein the dose of the felbamate or fluorofelbamate is between 100 and 200 mg.

23-36. (canceled)

37. A method for making the microparticles of claim 15 comprising mixing a solution of the felbamate or fluorofelbamate in a solvent into a solution of the surface modifying agent that is a non-solvent for the felbamate or fluorofelbamate to form a suspension of microparticles having an effective particle size less than 100 microns.

38. The method of claim 37, wherein the ratio of the non-solvent to the solvent is at least 20:1.

39. The method of claim 37, wherein the surface modifying agent is a surfactant and the surfactant concentration is at least 0.1 weight per volume.

40. The method of claim 37, wherein the surfactant stabilizes the suspension of microparticles by maintaining the mean particle size within 30%, preferably within 20%, more preferably within 10% of the initial mean particle size upon formation of the microparticles.

41. The method of claim 37, wherein the solvent is an organic solvent.

42. The method of claim 41, wherein the organic solvent is dimethyl sulfoxide.

43. The method of claim 41, wherein the organic solvent is glycerin heated to a temperature of at least about 90° C.

44. The method of claim 37, wherein the solvent is water heated to a temperature of at least about 50° C.

45. (canceled)

46. The method of claim 37, wherein the non-solvent is an aqueous solution of the surface modifying agent.

47. (canceled)

48. The method of claim 37, wherein a heated mixture of the solution of the agent and the aqueous solution of the surface modifying agent is cooled to effect formation of the microparticles.

49-59. (canceled)
